(19) United States
(12) Patent Application Publication
(10) Pub. No.: US 2011/0189051 A1
(43) Pub. Date: Aug. 4, 2011

(54) Reaction Cuvette Supply to an Automatic Analysis Machine

(76) Inventors: Jean-Francois Gelin, Creteil (FR); Julien Benizri, Vaires Sur Marne (FR); Dominique Casas, La Garenne-Colombes (FR)

(21) Appl. No.: 12/812,253

(22) PCT Filed: Jan. 27, 2009
(86) PCT No.: PCT/FR09/00088

§ 371 Date: Oct. 27, 2010

(30) Foreign Application Priority Data
Feb. 5, 2008 (FR) 08/00585

(51) Int. Cl. G01N 33/48

(52) U.S. Cl. 422/66; 422/63

(57) ABSTRACT

Device for supplying an automatic analysis machine with reaction cuvettes, comprising means (10, 20, 22) that define a housing for a cassette (14) containing superposed rows of reaction cuvettes, a buffer storage zone (16) for reaction cuvettes as they leave the cassette (14) positioned in its housing, and destacking means (18) that take up the cuvettes as they leave the buffer storage zone (16) and convey them one by one to a point at which the cuvettes enter the analysis machine.

The invention essentially relates to a device for supplying reaction cuvettes to an automatic analysis machine suitable for use particularly for determining the time required for the physical state of a medium to change and particularly for determining the blood clotting time.

From document EP-A-0325874, an analysis method and machine are known wherein a blood sample is deposited in a cuvette containing a ferromagnetic bead that can be displaced in periodic movement by means of an external magnetic field. The variations in the amplitude and/or frequency of the movements of the bead over time, which are representative of the changes of the physical state of blood, are detected by suitable means.

This machine may be supplied with the cuvettes in the manner described in documents WO 03/065047 and WO 03/065048, wherein cuvettes are connected to each other to form a continuous row, by means of a flexible film attached to the cuvettes so as to seal the openings thereof at least partially and prevent the beads from coming out of the cuvettes. The cuvette strip formed may may be rolled into a reel, said reel comprising for example around one thousand cuvettes and suitable for being loaded on an axis of rotation mounted for this purpose in a suitable compartment of the machine, said machine comprising means for unrolling the reel strip step by step and feeding the cuvettes one by one into the machine.

Operatives responsible for operating the machine sometimes encounter difficulties when changing the reel, as it is necessary to successively remove the empty reel, fit a full reel and insert the end of the film bearing the cuvettes correctly into the machine, so that the actuation means provided in the machine can feed the cuvettes one by one into the machine.

The shutdown of the analysis machine is mandatory for each change of reel and may be extended if the operatives have difficulties fitting a full reel.

Furthermore, it is costly to attach a film on the cuvettes to connect same in a continuous strip, particularly as this film is removed from the cuvettes when said cuvettes are used in the analysis machine.

The aim of the present invention is particularly that of avoiding these drawbacks of the prior art and providing a simple, effective and economical solution for the problem of supplying cuvettes to an analysis machine of the type described above.

To this end, it proposes a cassette for supplying reaction cuvettes to an automatic analysis machine, characterised in that it comprises:
two parallel side walls fixedly assembled with each other and comprising means for supporting rows of reaction cuvettes,
one closed end face, formed by edges of the side walls and preventing the cuvettes from coming out of the cassette,
and one open end face, opposite the closed end face and fitted with a sliding seal, movable between a cuvette exit position and a cuvette holding position.

The means for supporting the rows of cuvettes comprise ribs formed protruding on the inner faces of the side walls of the cassette and whereon the side edges of the cuvettes are supported.

Furthermore, the inner faces of the side walls of the cassette comprise fins parallel with the ribs for supporting the cuvettes and extending over same to close the open upper ends of the cuvettes supported by said ribs at least partially and prevent the beads contained in the cuvettes from coming out.

The cassette further comprises at least one pushing device formed by a transverse insert extending between the side walls and which is guided in translation on the means for supporting the cuvettes, said insert comprising side tabs or lugs extending through the slots of the side walls of the cassette to form means for actuating the insert from one end to the other of the cassette so as to move all the rows of cuvettes to the open end of the cassette simultaneously.

Such a cassette may contain for example approximately one thousand reaction cuvettes, which are simply arranged into rows stacked in the cassette and which are not connected to each other by a film or by any means.

The pushing device(s) provided in the cassette, which is/are guided in translation on means for supporting or guiding the cuvettes, make it possible to move all the cuvettes of the stacked rows simultaneously to the open end of the cassette.

This cassette may be produced economically in metal or injection-moulded or thermoformed plastic.

The invention also proposes an automatic analysis machine, particularly for determining the time required for the physical state of a medium to change, characterised in that it comprises:
means forming a housing to receive a cassette of the type described above,
means provided at one end of said housing and forming a cuvette buffer storage zone,
first transfer means for moving the cuvettes from the cassette placed in the housing to the buffer storage zone,
and second transfer means for moving the cuvettes from the buffer storage zone to a point at which the cuvettes enter the analysis machine.

In the machine according to the invention, the buffer storage zone enables the continuous operation of the machine when an empty cassette is removed and replaced by a full cassette. The quantity of cuvettes found in the buffer storage zone is provided to ensure machine operation for a much greater period of time than that required to change the cassette. When a new full cassette has been inserted in the machine and the buffer storage zone is cleared, it is refilled with cuvettes from said new cassette while the second transfer means supply the machine with cuvettes, as seen in more detail hereinafter.

For example, when the cassette contains approximately one thousand cuvettes divided into eighteen stacked rows, the buffer storage zone comprises eighteen stacked rows of nine cuvettes each, enabling continuous operation of the machine for approximately 30 minutes.

According to a further feature of the invention, the first cuvette transfer means comprise means for actuating the pushing device insert(s) guided in translation in the cassette, said actuation means comprising a carriage moved on a longitudinal guide rail by motor means and supporting at least one transverse cylindrical rod extending along the rows of cuvettes, perpendicular to these rows and supporting tappets to engage with the side lugs of the pushing device insert(s) to move same from one end of the cuvette rows to the other.

This cylindrical rod or each cylindrical rod is pivotably mounted about the axis thereof on the carriage, between a working position where the tappets are engaged with the side lugs of a pushing device insert and an idle position where the tappets are separated from the side lugs of said pushing device insert.

In this way, when the cylindrical rods are in the idle position thereof, they may be returned by the carriage to an initial starting position by being moved along a full cassette that has just been inserted in the housing and are then returned to the working position to engage with the side lugs of the pushing device plate(s) of said full cassette.

The rotation of the cylindrical rod(s) is advantageously controlled by cams supported by the or each cylindrical rod and which engage with abutments provided in the cassette housing.

The carriage supporting the cylindrical rod(s) may be rigidly connected to a belt guided on pulleys mounted in the cassette housing and one of which is rotated by motor means.

According to a further feature of the invention, the sealing plate of the open end of the cassette is moved between the two operating positions thereof by a movable member which is mounted at the entry of the buffer storage zone and is actuated by the carriage of the first transfer means, when the carriage arrives at the entry of the buffer zone.

In the cuvette exit position thereof, the sealing plate of the open end of the cassette engages with an abutment provided in the cassette housing to hold said cassette in position in the housing.

According to a further feature of the invention, the buffer storage zone comprises means for supporting the cuvettes extending from the ribs for supporting cuvettes of a cassette placed in said housing, to receive cuvettes from the rows of cuvettes contained in the cassette, and the second cuvette transfer means comprise at least one side comb wherein the teeth extend transversely between the means for supporting cuvettes in the buffer storage zone to push all the cuvettes from this zone simultaneously to a conveyor belt receiving the end cuvettes of the rows of the buffer storage zone and conveying same one by one to means for entering the cuvettes in the analysis machine.

In one preferred embodiment of the invention, the rows of cuvettes are stacked vertically in the cassette and in the buffer storage zone and the conveyor belt situated at the exit of the buffer zone is vertical and forms destacking means.

The comb(s) of the second cuvette transfer means is/are supported by a slide which is movable on the guide rail of the carriage of the first transfer means, said slide being pushed step by step to the exit of the buffer storage zone by the carriage of the first transfer means to clear said buffer storage zone and being returned by the carriage to the entry of the buffer storage zone when cleared.

In one preferred embodiment of the invention, said slide and carriage are rigidly connected to each other by means of magnetic attraction when the carriage is conveyed to the entry of the buffer storage zone, an abutment being provided at the entry of said zone to hold the slide and release same from the carriage when returned to the entry of the cassette housing.

The invention will be understood more clearly and further features, details and advantages thereof will emerge more clearly on reading the description hereinafter, with reference to the appended figures wherein:

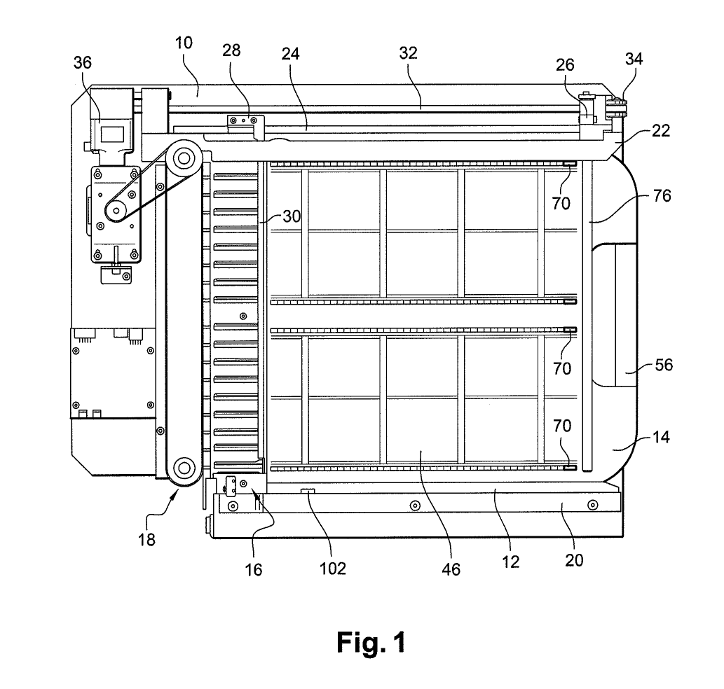

FIG. 1 is a schematic front view of a device for supplying reaction cuvettes to an analysis machine, a full cassette being in position in said device;

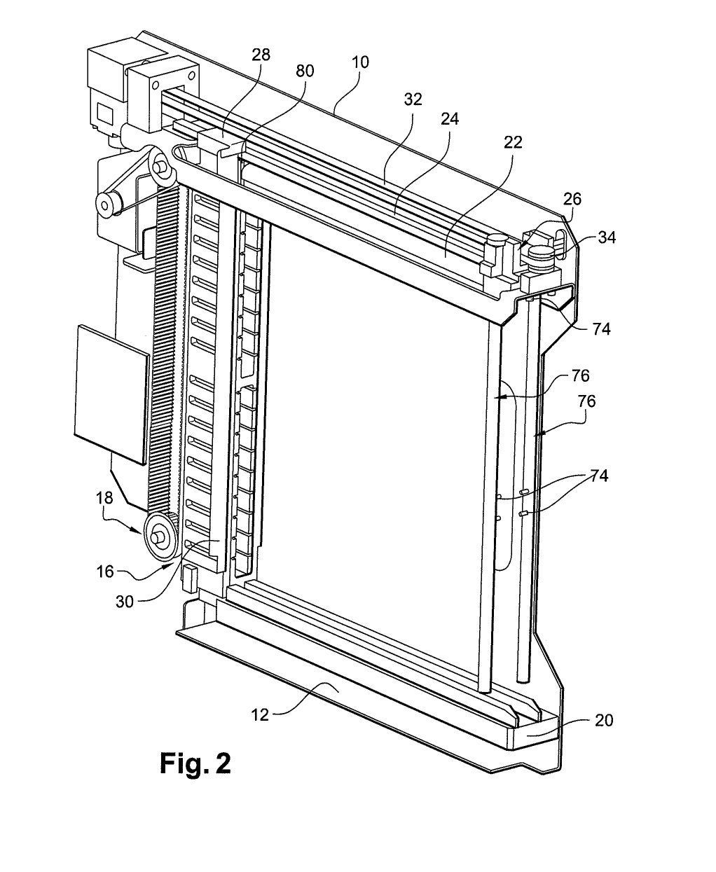

FIG. 2 represents the same supply device, without the cassette;

FIG. 3 is a schematic perspective view of a half-cassette;

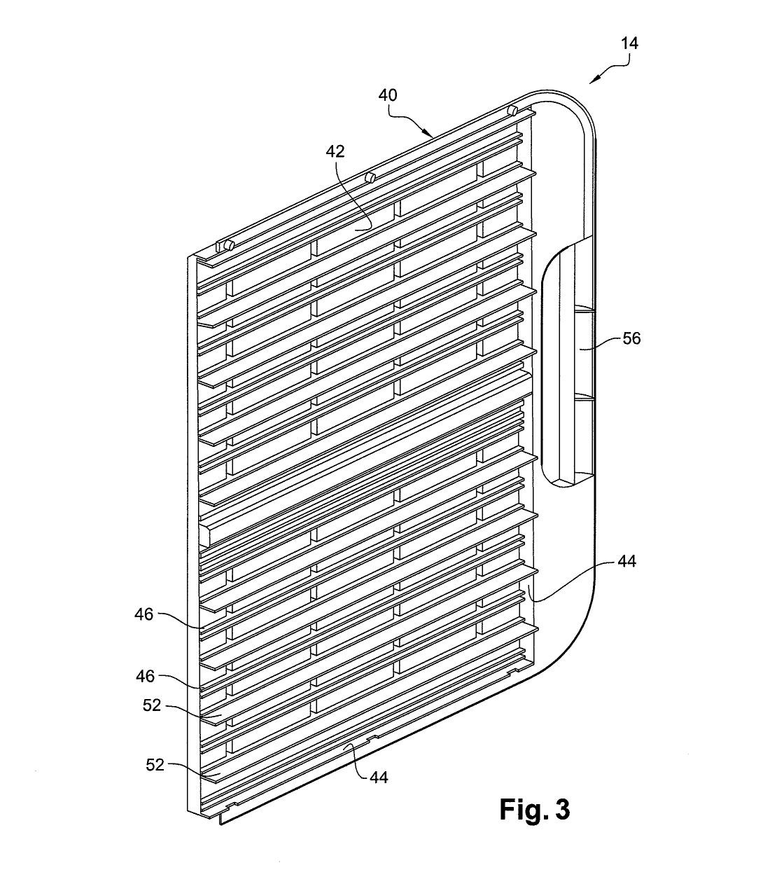

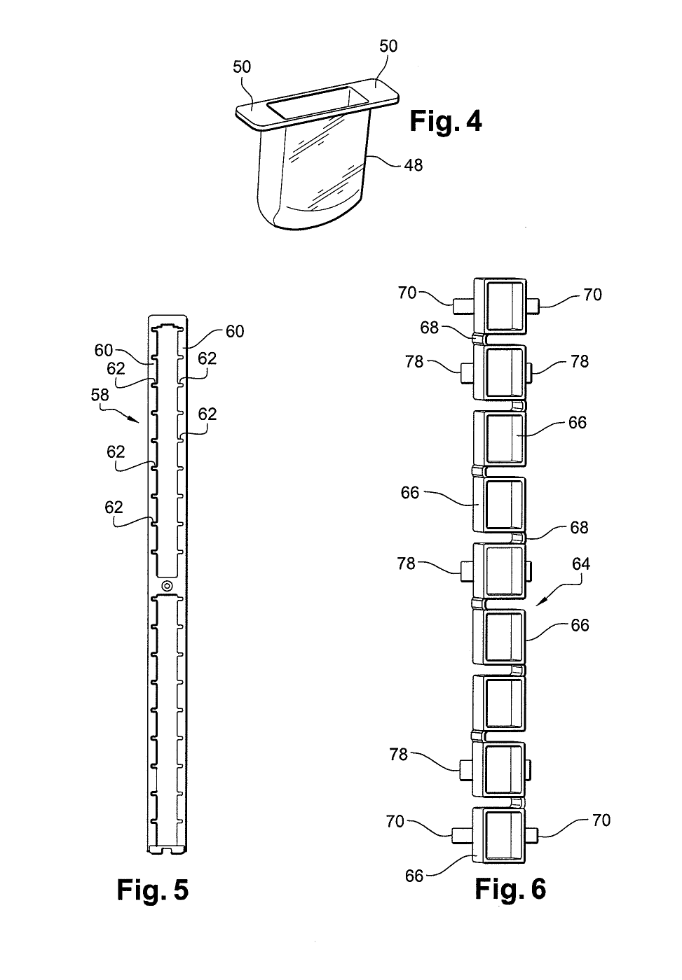

FIG. 4 is a larger-scale schematic perspective view of a reaction cuvette;

FIG. 5 is a front view of a sealing member of a cassette according to the invention;

FIG. 6 is a schematic perspective view of a pushing device of the cassette;

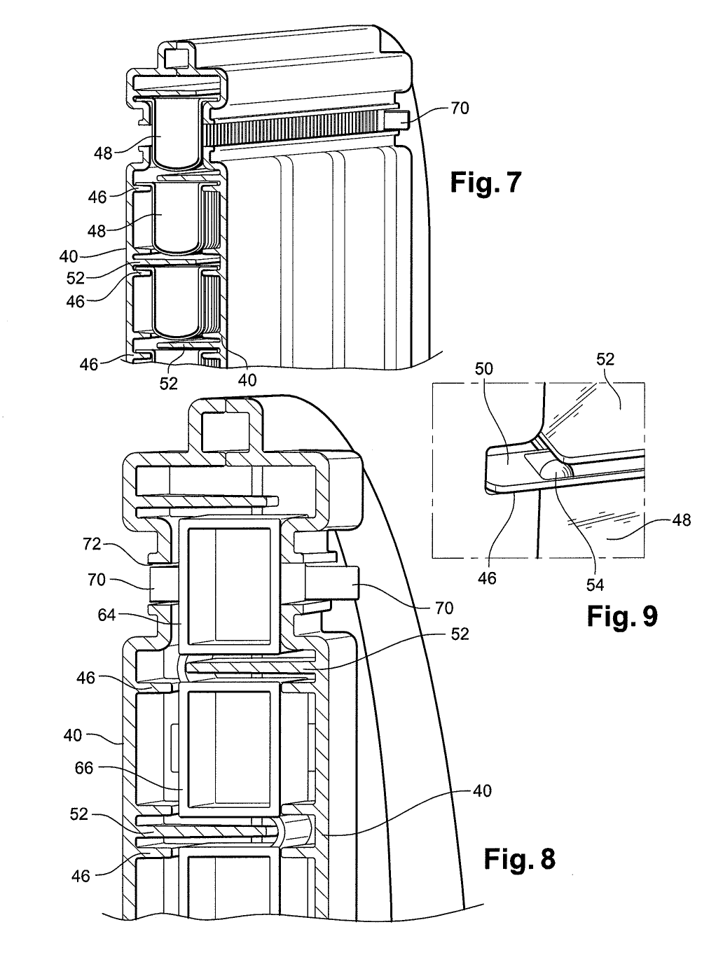

FIG. 7 is a partial schematic perspective and cross-sectional view representing the arrangement of the cuvettes in a cassette according to the invention;

FIG. 8 is a partial schematic perspective view representing a pushing device in position in the cassette;

FIG. 9 is a larger-scale schematic perspective view representing means for holding the beads in the cuvettes in a cassette according to the invention;

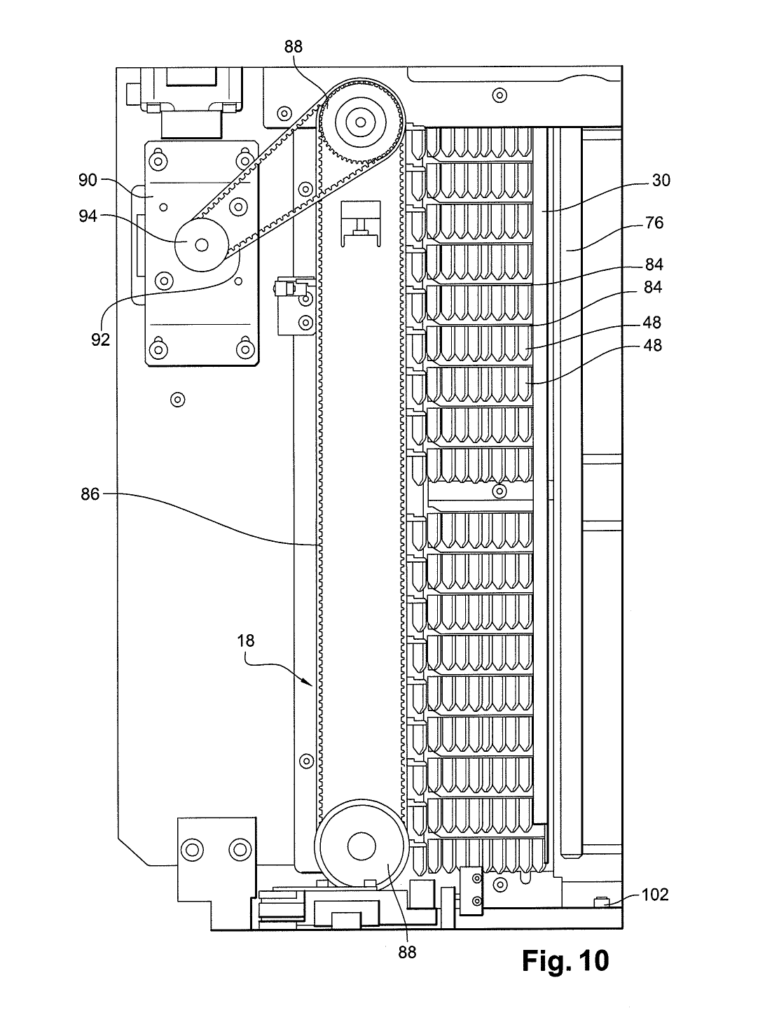

FIG. 10 is a larger-scale schematic front view of the buffer storage zone and the destacking means;

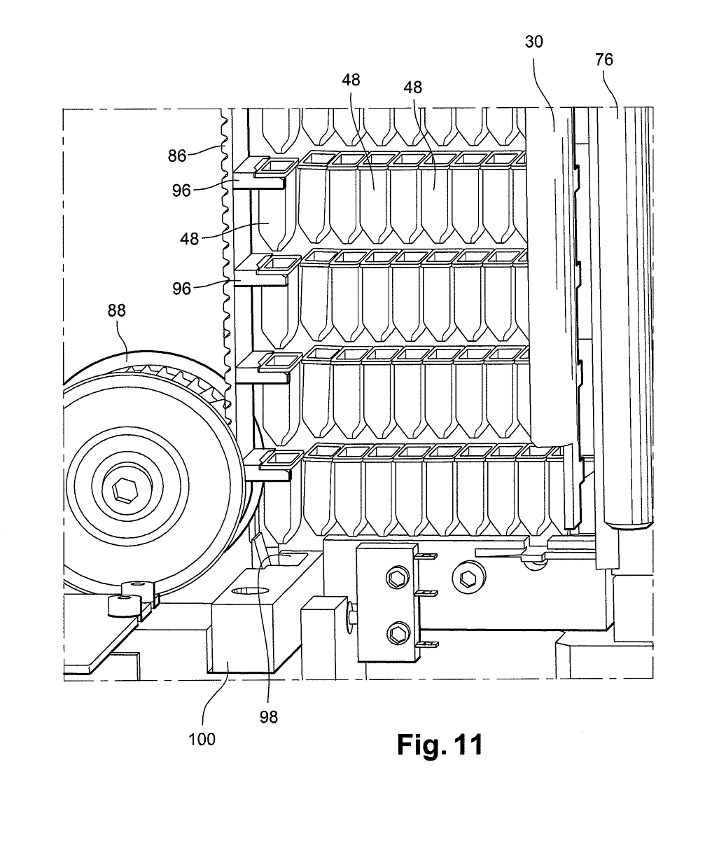

FIG. 11 is a larger-scale partial schematic view of the destacking means exit.

The device for supplying reaction cuvettes represented in FIGS. 1 and 2 is part of an automatic analysis machine, particularly for determining the time required for the physical state of a medium to change, and essentially comprises a plate 10 to be attached on the machine and supporting means 12 forming the housing of a cassette 14 containing an ordered arrangement of reaction cuvettes, means 16 forming a reaction cuvette buffer storage zone, and means 18 for conveying reaction cuvettes one by one to a point at which the cuvettes enter the analysis machine.

The means 12 forming the housing for receiving a cassette 14 comprise two vertically stacked horizontal rails 20, 22 wherein the lower and upper horizontal edges of the cassette 14 are guided, foolproof means being provided on one of the guide rails and on the corresponding edge of the cassette to prohibit back-to-front assembly of the cassette 14 in the housing thereof.

A cassette presence detector 102 in the housing may also be provided in the front part of the lower rail 20, to detect whether a cassette 14 has been fitted the correct way in the housing and has been pushed to the entry of the buffer storage zone 16.

The upper part of the housing also comprises a horizontal rail 24, extending above the upper guide rail 22 all along the cassette housing and also all along the buffer storage zone 16 and the means 18 for conveying the cuvettes to the entry of the machine.

This rail 24 is used for supporting and guiding a carriage 26 supporting the means for moving the reaction cuvettes in the cassette 14 to the buffer storage zone 16, and a slide 28 supporting vertical combs 30 for moving the reaction cuvettes in the buffer storage zone 16.

The carriage 26 is attached to a horizontal belt 32 running on two opposite pulleys 34, only one of which situated at the rear end of the cassette housing can be seen in FIGS. 1 and 2, the other pulley being rotated by motor means 36 to move in a to-and-fro movement, by means of the belt 32, the carriage 26 between the rear end of the cassette housing and the front end of the buffer storage zone 16, as described in more detail hereinafter.

The cassette 14 containing an ordered arrangement of reaction cuvettes will now be described with reference to FIGS. 3 to 9. This cassette has a rectangular parallelepipedic shape and is formed by juxtaposing and attaching two half-shells 40, one of which is represented in detail in FIG. 3, said two half-shells being made for example of a plastic such as injection-moulded polystyrene.

Each half-shell 40 essentially comprises a plane wall 42 forming one of the large side faces of the cassette and a peripheral edge 44 engaging with the edge 44 of the other half-shell to form the small horizontal and vertical faces of the cassette when both half-shells are assembled.

Longitudinal horizontal ribs 46 are formed protruding on the inner faces of the half-shells 40 to support the rows of reaction cuvettes 48 as represented in FIG. 4, each cuvette comprising two opposite side edges 50 at the open upper edge thereof, whereby the cuvette rests on a rib 46 of a half-shell 40 and on a rib 46 situated at the same level at the other half-shell 40.

The arrangement of rows of cuvettes 48 on the ribs 46 of the half-shells was illustrated schematically in FIG. 7.

Horizontal longitudinal fins 52 are also formed protruding on the inner faces of the half-shells 42, alternating with the ribs 46 such that each fin 52 extends slightly above a cuvette 48 and seals the open upper end of said cuvette incompletely but sufficiently to prevent a ferromagnetic bead 54 contained in the cuvette from coming out of same, as illustrated schematically in FIG. 9.

It is possible for example, as represented in FIGS. 7 and 8, to form said fins 52 alternating on one half-shell 40 for one row of cuvettes 48 and on the other half-shell 40 for the upper or lower row of cuvettes 48.

The rear vertical end of the cassette 14 is closed by the edges 44 of the half-shells 40 and comprises a handle 56 for convenient handling of the cassette. The front vertical end of the cassette is open, to enable the rows of cuvettes 48 to exit to the buffer storage zone 16 when the cassette is in position in the housing of the supply device, and is fitted with a seal 58 (FIG. 5) formed from an elongated frame wherein the dimensions correspond to those of the front vertical end of the cassette, the vertical sides 60 of said frame comprising notches 62 on the inner edges thereof corresponding to the edges 50 of the reaction cuvettes 48 contained in the cassette.

The seal 58 is guided in vertical translation between the two half-shells 40 and is mounted, when the cassette is filled with reaction cuvettes, in a closing position prohibiting the exit of the cuvettes. This makes it possible to handle the cassette while preventing the cuvettes from falling, regardless of the orientation of the cassette.

To enable the exit of the cassette when the cassette has been positioned in the housing of the supply device, the seal 58 may be moved by a member described in more detail hereinafter such that the notches 62 of the edges 60 thereof are aligned with the edges 50 of the cuvettes 48 resting on the ribs 46 of the half-shells 40.

A pushing device 64 represented in FIG. 6 is mounted inside the cassette, at the rear end thereof, to push the rows of cuvettes contained in the cassette to the front end thereof.

This pushing device 64 extends on the entire height of the cassette or, in the example represented, on half of the height thereof, two pushing devices 64 being positioned aligned with each other at the rear end of the cassette.

Each pushing device 64 comprises a series of rectangular or square blocks 66, the number whereof corresponds to the number of rows of cuvettes that the pushing device 64 needs to move inside the cassette and wherein the shape corresponds to the contour of a reaction cuvette. These various blocks 66 are connected to each other by jumpers 68 arranged in alternation on one vertical side of the pushing device 64 and on the other vertical side thereof, to enable mounting of the pushing device on the fins 52 formed in alternation on the two half-shells 40, as represented schematically in FIG. 8.

This configuration enables satisfactory support and guidance of each pushing device 64 inside the cassette 14.

The end blocks 66 of the pushing device 64 bear side lugs 70 extending through the slots 72 of the side walls of the half-shells 40 and intended to engage with tappets 74 of two vertical rods 76 supported by the carriage 26, said two rods 76 extending on either side of the cassette 14 such that the tappets 74 thereof can abut against the lugs 70 of the pushing devices 64 and move same in the direction of the buffer storage zone 16.

The rods 76 are pivoting on a quarter turn about the axis thereof on the carriage 26, between two positions where the tappets 74 are respectively perpendicular to the walls 42 of the cassettes and facing each other, and where said rods are parallel with the walls 42, respectively.

Cams supported by the upper ends of the rods 76 bear on abutments provided at the ends of the travel of the carriage 26 to pivot the rods 76 from one of the positions thereof to the other and conversely.

As represented in FIG. 6, other blocks 66 of the pushing device 64 may support side blocks 78 of a smaller size to the lugs 70 of the end blocks 66.

For the assembly of the cassette, the pushing devices 64 are fitted on a half-shell 40, at the rear end of said half-shell, and the second half-shell is mounted on the first, the mounting taking place for example by snap fastening such that the cassette is no longer dismountable without breaking some of the parts thereof. The assembled cassette may be filled with reaction cuvettes, until completely full. In the example of an embodiment represented, the cassette 14 comprises eighteen stacked rows of fifty-six cuvettes, i.e. a total of 1008 cuvettes each containing one bead 54.

When the cassette is filled with cuvettes, the seal 58 is fitted at the front end of the cassette in a position where it prohibits the exit of the cuvettes. It can be seen in FIG. 5 that the vertical ends of the seal 58 are not identical, such that the seal can act as a foolproof device enabling the full insertion of the cassette into the housing of the supply device in FIGS. 1 and 2 when the cassette is positioned correctly and prohibiting this full insertion when the cassette is back-to-front.

The entry of the buffer storage zone 16 comprises a vertical member 80 guided in vertical translation on said end of the zone 16 and engaging with the seal 58 of the front end of the cassette to move said seal between the cuvette exit opening and closing positions thereof, said movable member 80 being in turn moved in translation by the carriage 26 by means of a ramp system engaging with the movable member 80 to move same vertically in one direction and in the other. In this way, when the carriage 26 reaches the entry of the buffer storage zone 16, it lowers the member 80, which places the seal 58 in a position prohibiting the exit of the cuvettes from the cassette. When the carriage 26 is returned to the rear position represented in FIGS. 1 and 2, to unload a full cassette that has just been placed in the housing of the supply device, the movable member 80 is raised again by the carriage ramp system, placing the seal 58 of the new cassette in a position enabling the exit of the cuvettes. The seal 58 is then in a high position wherein the upper end thereof can engage with an abutment supported by the front end of the buffer storage zone 16, locking the cassette 14 in the housing thereof of the supply device and prohibiting the removal of same from said housing.

The buffer storage zone 16 in turn comprises a series of horizontal longitudinal ribs 84 for supporting the edges 50 of the reaction cuvettes 48, extending from the ribs 46 of the cassette 14. The buffer storage zone 16 may thus comprise eighteen vertically stacked rows of reaction cuvettes, each row comprising nine reaction cuvettes for example.

The combs 30 supported by the slide 28 extend transversely behind the last reaction cuvette 48 of each row and make it possible to move the reaction cuvettes 48 forwards in the buffer storage zone 16 when the slide 28 is moved forwards by said carriage 26.

At the front end of the buffer storage zone 16, means 18 are situated, comprising a vertical conveyor destacker formed from a synchronous belt 86 running on two vertically aligned pulleys 88, the upper pulley 88 being rotated by an electric motor 90 and by a synchronous belt 92 running on a pulley rigidly connected to the upper pulley 88 and on a pulley 94 rigidly connected to the exit shaft of the motor 90. The vertical ply of the belt 86, extending along the exit of the buffer storage zone 16, bears U-shaped supporting members 96 facing the zone 16 and each intended to receive one reaction cuvette 48 as represented in FIG. 11.

The step-by-step lowering of said ply of the belt 86 makes it possible to unload the reaction cuvettes one by one in a housing 98 of a reception unit 100 which is then moved by suitable means to a point at which the reaction wells enter the automatic analysis machine.

The device for supplying reaction cuvettes described above operates as follows:

When a cassette 14 positioned correctly in the rails 20, 22 of the housing thereof in the supply device has been cleared of the reaction cuvettes thereof, the carriage 26 bearing cylindrical rods 76 is situated at the front of said housing and just at the entry of the buffer storage zone 16 as represented in FIGS. 10 and 11. In this position, the buffer storage zone 16 is entirely full of reaction cuvettes 48. When all the reaction cuvettes supported by the supporting members 96 of the belt 86 have been unloaded in succession in the reception unit 100 and conveyed to the entry of the automatic analysis machine, the motor 90 is commanded to raise the belts 86 and the supporting members 96 to an initial position represented in FIG. 10 where each supporting member 96 extends from a row of reaction cuvettes 48 of the buffer storage zone 16. The carriage 26 is then moved one step forwards and pushes the slide 28 one step towards the exit of the buffer storage zone 16, which unloads one reaction cuvette 48 onto each supporting member 96 of the belt 86. The motor 90 is then commanded again to lower the supporting members 96 on one step and unload one reaction cuvette 48 in the housing 98 of the reception unit 100.

The process may continue until all the reaction cuvettes contained in the buffer storage zone 16 have been unloaded one by one into the reception unit 100, ensuring continuous operation of the analysis machine for approximately 30 minutes.

Meanwhile, the empty cassette may be removed from the housing thereof in the supply device and replaced by a cassette 14 filled with reaction cuvettes. For this, it is simply necessary to pull the handle 56 of the empty cassette and extract same from the rails 20, 22 and take a cassette 14 filled with reaction cuvettes by the handle 56 thereof and insert same into the rails 20, 22 of the supply device housing.

The foolproof means prohibit positioning the cassette 14 back-to-front. When the cassette 14 has been inserted into the housing thereof up to the entry of the buffer storage zone 16, a detector 102, visible in FIGS. 1 and 10, confirms the correct assembly of the cassette in the supply device housing and enables the control of the movement of the carriage 26 by the motor means 36.

The carriage 26, which had been moved forward to the exit of the buffer storage zone 16 to empty same of the cuvettes thereof, is returned to the rear by the belt 32 to the rear end of the cassette 14 in the position represented in FIGS. 1 and 2. During this return movement, it commands the movement of the seal 58 of the cassette to position same in a position enabling the exit of the reaction cuvettes and locking the cassette in the housing thereof of the supply device.

Furthermore, during this return movement, the cylindrical rods 76 supported by the carriage 26 are in an angular position about the axis thereof where the tappets 74 extend parallel with the rails 20 and 22, making it possible to move the rods 76 along the side walls 42 of the cassette 14 without interfering with the lugs 70 of the pushing devices 64, found in the rear position in the cassette as represented in FIG. 1.

When the carriage 26 reaches the end of the travel in the rear position, cams supported by the upper ends of the rods 76 come into contact with corresponding abutments supported by the rail 22 and pivot the rods 76 by a quarter-turn such that the tappets 74 extend towards each other and perpendicular to the rails 20 and 22, as represented in FIG. 2. In this position, the tappets 74 are applied on the lugs 70 of the pushing devices 64 when the carriage 26 is moved forwards.

The arrival of the carriage 26 in the rear position thereof, after opening the exit of the cassette by the seal 58 and locking of the cassette in the housing thereof of the supply device, enables the return of the carriage 26 to the front position thereof. This movement of the carriage 26 causes a forward movement of the rows of cuvettes contained in the cassette 14 actuated by the pushing devices 64, until the buffer storage zone 16 is filled with reaction cuvettes again. Then, when all the reaction cuvettes supported by the supporting members 96 of the belt 86 of the destacking means 18 have been deposited in succession in the reception unit 100, and when the supporting members 96 have been returned in line with the rows of reaction cuvettes of the zone 16, further advancement of the carriage 26 by one step makes it possible to refill the supporting members 96, and so on.

When the carriage 26 reaches the entry of the buffer storage zone 16, the cams supported by the upper ends of the cylindrical rods 76 press on fixed abutments rotating the rods 76 by a quarter turn about the axis thereof to orient the tappets 74 parallel with the rails 20 and 22. The abutment of the carriage 26 on the slide 28 makes it possible to move same forwards step by step, to clear the buffer storage zone 16. For this purpose, permanent magnets may be mounted on the carriage 26 and/or on the slide 28 to rigidly connect same by means of magnetic attraction when in contact with each other. When the slide 28 has been returned by the carriage 26 to the entry of the buffer storage zone 16, it is held by a fixed abutment preventing same from moving further to the rear when the carriage 26 is returned to the position in FIG. 1.

Furthermore, the arrival of the carriage 26 in the front position thereof at the entry of the buffer storage zone 16 also commands the movement of the seal 58 in the position thereof for closing the exit of the cassette 14 and unlocking said cassette, which may then be removed from the housing thereof of the supply device.

1. Cassette for supplying reaction cuvettes to an automatic analysis machine, comprising;
two parallel side walls fixedly assembled with each other and comprising means for supporting rows of reaction cuvettes,
one closed end face, formed by edges of the side walls and preventing the cuvettes from coming out of the cassette,
and one open end face, opposite the closed end face and fitted with a sliding seal, movable between a cuvette exit position and a cuvette holding position.

2. Cassette according to claim 1, wherein the means for supporting the rows of cuvettes comprise ribs formed protruding on the inner faces of the side walls of the cassette and whereon the side edges of the cuvettes are supported.

3. Cassette according to claim 2, wherein the inner faces of the side walls of the cassette comprise fins parallel with the ribs for supporting the cuvettes and extending over same to close the open upper ends of the cuvettes supported by said ribs at least partially and prevent the beads contained in the cuvettes from coming out.

4. Cassette according to claim 1, comprising at least one pushing device formed by a transverse insert extending between the side walls and which is guided in translation on the means for supporting the cuvettes, said insert comprising side lugs or tabs extending through the slots of the side walls of the cassette to form means for actuating the insert from one end to the other of the cassette so as to move all the rows of cuvettes to the open end of the cassette simultaneously.

5. Cassette according to claim 1, wherein the closed end of the cassette comprises a handle (56) for handling.

6. Cassette according to claim 1, said cassette being made of metal or injection-moulded or thermoformed plastic.

7. Automatic analysis machine, particularly for determining the time required for the physical state of a medium to change, comprising means for supplying reaction cuvettes, said supply means comprising:
means forming a housing to receive a cassette according to claim 1,
means provided at one end of said housing and forming a cuvette buffer storage zone,
first transfer means for moving the cuvettes from the cassette placed in the housing to the buffer storage zone,
and second transfer means for moving the cuvettes from the buffer storage zone (16) to a point at which the cuvettes enter the analysis machine.

8. Machine according to claim 7, wherein means for actuating a pushing device in the cassette comprise a carriage, moved on a longitudinal guide rail by motor means, said carriage supporting at least one transverse cylindrical rod extending along the rows of cuvettes, perpendicular to these rows and supporting tappets to engage with the side lugs of the pushing device to move same from one end of the cuvette rows to the other and release the cuvettes from the cassette.

9. Machine according to claim 8, wherein the or each cylindrical rod is pivotably mounted about the axis thereof on the carriage, between a working position where the tappets are engaged with the side lugs of the pushing device and an idle position where the tappets are separated from the side lugs of said pushing device.

10. Machine according to claim 8, wherein the or each cylindrical rod support cams engaging with fixed abutments of the housing of the cassette, to pivot the rod from one of the positions thereof to the other and conversely.

11. Machine according to claim 8, wherein the means for actuating the carriage comprise a belt guided on pulleys one of which is rotated by motor means.

12. Machine according to claim 7, wherein the sealing plate of the exit of the cassette is moved between the two operating positions thereof by a movable member which is mounted at the entry of the buffer storage zone and is actuated by the carriage of the first transfer means.

13. Machine according to claim 11, wherein the sealing plate engages in the cuvette exit position thereof with an abutment provided in the cassette (14) housing to hold said cassette in position in the housing.

14. Machine according to claim 7, wherein the buffer storage zone comprises means for supporting the cuvettes extending from the means for supporting cuvettes of a cassette placed in said housing, to receive cuvettes from the rows of cuvettes contained in the cassette, and in that the second cuvette transfer means comprise at least one side comb wherein the teeth extend transversely between the means for supporting cuvettes in the buffer storage zone to push all the cuvettes from this zone simultaneously to a conveyor belt receiving the end cuvettes of the rows of cuvettes of the buffer storage zone and conveying same one by one to means for entering the cuvettes in the analysis machine.

15. Machine according to claim 7 wherein, in the cassette and in the buffer storage zone, the rows of cuvettes are stacked vertically and in that the conveyor belt situated at the exit of the buffer zone is vertical.

16. Machine according to claim 15, wherein the belt of the conveyor supports a series of cuvette supporting members, the number of these supporting members being equal to the number of rows of cuvettes in the buffer storage zone.

17. Machine according to claim 14, wherein the comb of the second transfer means is/are supported by a slide which is movable on the guide rail of the carriage of the first transfer means, said slide being pushed step by step to the exit of the buffer storage zone by the carriage of the first transfer means to clear said buffer storage zone and being returned by the carriage to the entry of the buffer storage zone when cleared.

18. Machine according to claim 17, wherein the slide and carriage are rigidly connected to each other by means of magnetic attraction when the carriage is conveyed to the entry of the buffer storage zone, an abutment being provided at the entry of said zone to hold the slide and release same from the carriage when said carriage is returned to the entry of the cassette housing.
